(19) United States
(12) Patent Application Publication
(10) Pub. No.: US 2005/0072421 A1
(43) Pub. Date: Apr. 7, 2005

(54) Apparatus, system and method for positive confirmation of inhaled drug delivery by attenuation at point-of-use

(75) Inventors: Julie Suman, Ellicott City, MD (US); Shailaja Somaraju, Catonsville, MD (US)

Correspondence Address: VENABLE, BAETJER, HOWARD AND CIVILETTI, LLP, P.O. BOX 34385, WASHINGTON, DC, 20043-9998, US

(73) Assignee: NEXT BREATH, LLC

(21) Appl. No.: 10/677,485

(22) Filed: Oct. 3, 2003

(51) Int. Cl. A61M 11/00

(52) U.S. Cl. 128/200.23

(57) ABSTRACT

A system, method and apparatus for providing positive confirmation of inhaled drug delivery by light or radiation attenuation at point-of-use is disclosed, including a tube adapted to be coupled at an outlet to an inhaler; a light or radiation source coupled to the tube; and a light or radiation detector coupled to the tube, where the source transmits light or radiation along a path intersecting a path of an emission such as a drug dose emitted from the inhaler, and where the detector receives the light or radiation from the source.

### BACKGROUND OF THE INVENTION

1. Field of the Invention

The present invention relates generally to drug delivery systems, and more particularly to improvements to metered dose inhaler drug delivery systems.

2. Related Art

Conventional aerosol inhalation through the mouth as illustrated for example in FIG. 1 provides a convenient, non-invasive way to administer life-enhancing and life-saving drugs to pediatric, adult and geriatric patients. Therapeutic agents can be administered directly to their sites of action in the lung, or the lung can merely serve as a point of entry into the bloodstream. Examples of drugs given by inhalation to elicit a local response include bronchodilators, anti-inflammatory corticosteroids and anti-cholinergic agents, primarily used to treat respiratory diseases including, asthma, chronic bronchitis, emphysema and cystic fibrosis. A small percentage of today's locally acting inhaled drugs are also given for their local anesthetic, antibiotic and antiviral properties—an application likely to become more common in light of recent events. Nicotine, administered in smoking cessation therapy, is given as an inhalation aerosol for systemic absorption for which the ultimate target is the brain. At least some next generation inhaled drugs are intended to reach organs other than the lung, and can include, e.g., proteins, peptides, hormones and gene therapy vectors—intended to treat heart disease, osteoporosis, diabetes and several reproductive disorders.

Conventionally, pharmaceutical companies prefer to administer drugs as tablets or capsules. However, with the advent of biotechnology-derived drugs the oral route is not always viable. Reasons include metabolic degradation, low solubility and/or poor absorption. The pulmonary blood circulation, into which inhaled drugs are rapidly absorbed, largely by-passes liver and gut wall metabolism, thus offering an alternative point of entry for drugs susceptible to first pass hepatic or gastrointestinal metabolism or stomach acid. High molecular weight proteins and peptides rendered biologically inactive following oral administration, are know to retain activity following inhalation.

Conventionally, when a drug could not be used orally, the drug was injected. Such an approach is only viable for life-saving treatments or hospital administration, and will not be warranted by non-immanently life threatening indications sought by many biotechnology companies for their new chemical entities. Pulmonary inhalation offers an alternative to traditional needle sticks. Avoiding the need for parenteral administration, pulmonary inhalation is widely perceived as the key to bringing fragile biologicals to the US market—and several highly successful companies have been formed on this assumption. However, patients will not benefit from successful biotechnology based drug discovery, and the biotechnology industry will not be commercially successful if industry products are inconvenient to use. For these reasons interest in inhalation drug delivery is increasing dramatically, as indicated by numerous patent filings and collaborative agreements between niche pulmonary delivery companies and Big Pharma. For example, Nektar Therapeutics Inc., of San Carlos, Calif. U.S.A. in collaboration with Pfizer of Groton, Conn. U.S.A., has obtained patents related to device design, formulation and excipients (ingredients other than the active drug) to facilitate diabetes treatment using dried particles of inhaled insulin administered through a dry powder inhaler (DPI). Aradigm, Inc., of Hayward, Calif. U.S.A. has intellectual property related to actuator design, small reproducible particle generation, and strategies to coordinate drug release relative to patient inspiration, and is focused on pain management by inhaled aqueous droplets of morphine. Companies such as 3M, of Saint Paul, Minn. U.S.A. believe they can administer proteins using the familiar pressurized metered dose inhaler (MDI) platform.

In addition to avoiding needles and maximizing absorption, drugs given by inhalation achieve local pharmacological effects, reach the diseased site almost instantaneously and, in many cases, elicit a rapid onset of action. In the treatment of acute and chronic asthma, administration of albuterol available from GlaxoSmithKline of Research Triangle Park, N.C. U.S.A., via inhalation, is known to elicit faster and longer-lasting bronchodilation than intravenous administration of a similar dose. Patients can breath easier in as little as 15 seconds after inhaling some bronchodilators. Similar findings have been reported for inhaled morphine in the management of cancer pain. In both cases, the patient quickly knows if their drug is working, and can take corrective action if it is not. Unfortunately, not all drugs that are inhaled have immediate, perceptible effects, and in most cases, the patient will not immediately recognize that a dose was not properly administered. Existing conventional inhalers cannot positively identify whether a drug administered by the inhaler was properly dispensed or not.

What is needed then, is an apparatus, system, and method that addresses administration of an inhaled drug, which conventionally does not notify a patient as to whether a dose has been correctly dispensed. Almost all of today's drugs and most of those currently under development have this unmet need. For example, a diabetic is not immediately aware that the diabetic's inhaled insulin dose was not properly administered. The diabetic is not aware since the delivery system is specifically designed to avoid throat deposition. So administration of the drug does not produce a sensation or biological effect that is immediately perceptible to the patient. As another example of this unmet need, a patient who does not correctly inhale a contraceptive steroid does not know anything went wrong with dispensing until the patient becomes pregnant. Yet another example of the unmet need is a patient with a bacterial or viral respiratory tract infection who does not know that misinhaled medication never reached the patient's lung until the infection becomes more severe. Thus, many drugs that could potentially elicit a beneficial biological response following inhalation, may not become successful drug products.

Inhalation drug delivery devices differ from other dosage forms because the benefit derived from the drug in the device depends as much on the patient's technique when using it, as on the effectiveness of the drug itself. Among the several types of inhalation aerosols conventionally available, metered dose inhalers (MDIs) are widely used to administer drugs to the lungs of ambulatory patients, primarily due to the MDIs' apparent ease of use, low cost and portability. However, research shows that over 50% of patients use inhalers incorrectly due to an inability to coordinate inspiration with inhaler actuation or due to exhaling when the drug is released. Such a lack of coordination, or performing the wrong breathing maneuver often results in poor disease management which is further exacerbated in the very young, very old and the infirm. A conventional improvement includes MDI dosing in conjunction with a spacer, which is now recommended for treating young children during acute episodes to partially mitigate against this problem.

Pharmaceutical companies have not traditionally focused on compliance issues, and health care providers typically give minimal guidance to patients on the correct way to utilize their inhaler. Several studies site this lack of understanding as the primary reason for inadequate outcomes from inhaler therapy and patient non-compliance. Furthermore educators such as, e.g., physicians, nurses and other health care professionals, often propagate incorrect inhaler techniques to their patients. If patients and their doctors have trouble with the simplest pressurized inhaler, one can anticipate that the patients will encounter more difficulties with today's more complex devices, and those in the development and testing pipeline. The effect of these technique failures will be magnified by the use of increasingly potent biotechnology-derived drugs.

Increased awareness of the importance of patient training for successful aerosol therapy, accompanied by the need to keep health care costs down, has driven development of a wide variety of auxiliary products in respiratory care. In an attempt to compensate for poor patient technique and to increase the dose of drug delivered to the lungs, several spacer devices and holding chambers have been developed for use in conjunction with MDIs. The space devices serve to reduce undesirable throat deposition of drug, thus minimizing side effects such as oral candidiasis and dysphonia due to inhaling steroids such as beclomethasone. Studies have shown that many asthmatic patients who have coordination problems with their inhalers benefit from using such devices. Conventional spacers and holding chambers have partially addressed the patient coordination problem. However, conventional inhalation devices do not provide positive confirmation that a dose left the device and entered the patient.

### SUMMARY OF THE INVENTION

An exemplary embodiment of the present invention is directed to an apparatus, system and method for providing an optical sensor to give positive confirmation that a drug has been inhaled by a patient. The apparatus can provide positive confirmation of inhaled drug delivery by detecting light or radiation attenuation at point-of-use caused by emitting an inhaled drug through a light or radiation beam. An exemplary embodiment of the apparatus of the present invention can include a tube adapted to be coupled at an outlet to an inhaler; a light source coupled to the tube; and a detector coupled to the tube, where the light source transmits light along a path intersecting a path of a drug dose emitted from the inhaler, and where the detector receives the light from the light source. Alternatively, one could use technology similar to that used in a smoke detector.

In one exemplary embodiment of the apparatus of the present invention the light transmitted can be a visible spectrum light, outside of the visible spectrum, or other radiation. For example, other radiation could include an alpha emitted such as, e.g., americium or palladium.

In another exemplary embodiment of the apparatus of the present invention the outlet can be a dry powder inhaler (DPI) outlet.

In yet another exemplary embodiment of the apparatus of the present invention the apparatus can further include an inhaler. In one embodiment, the inhaler can include a dry power inhaler (DPI) or a pressurized metered dose inhaler (MDI).

In another exemplary embodiment of the apparatus of the present invention the tube includes a cylinder having a geometrically shaped cross-section comprising at least one of: a cylinder of circular cross-section; a cylinder of rectangular cross-section; and a cylinder of square cross-section.

In another exemplary embodiment of the apparatus of the present invention the apparatus further includes a deflector for deflecting emitted aerosol from the light source or the detector to avoid aerosol accumulation.

The present invention allows safer use of extremely potent agents than with conventional actuator from complex delivery systems with a higher degree of confidence that a patient received the drug.

The present invention is beneficial for inhalation of any drug, but especially drugs that do not cause an immediately discernible physiological effect in a patient.

When utilized during clinical trials, the device of the present invention minimizes therapeutic failures from being erroneously attributed to a drug, when actually the device or patient-misuse is the cause of the problem.

The device of the present invention also provides a tool to train patients to use all inhalation devices correctly. An exemplary embodiment of the present invention includes a sensor that can provide a positive or negative indication whether a dose was properly inhaled. In another exemplary embodiment, the sensor can provide a quantitative measurement of the amount of drug inhaled.

A feature of an exemplary embodiment of the present invention can include a universal, compact, low cost drug delivery medical device. The device can be approvable by the US Food and Drug Administration's 510(k) process. The device can be added to all pulmonary drug delivery systems to provide a positive confirmation that a drug left the drug delivery device and entered the patient.

An exemplary embodiment includes an inexpensive and compact optical or radiation-based sensing technology capable of detecting low concentrations of airborne particles such as, e.g., a sensor capable of detecting aerosol particles exiting a range of pulmonary delivery systems under inspiratory flow rate conditions that occur when pediatric, adult and geriatric patient groups use inhalation delivery systems. The sensor detects common modes of device misuse. Maximum interior dimensions can be adapted such that the sensor mounting assembly does not alter performance of an original inhaler to which the assembly can be coupled.

The present invention includes various features providing several advantages. Some of the advantages include, for example, a benefit to a patient and a physician from knowing that the patient actually inhaled the drug that the physician intended for the patient to receive each time the patient attempts to take a dose. Medical insurance providers benefit because the number of therapeutic failures due to inhaler misuse would be reduced, with associated cost savings in treatment costs, as well as missed days at work and school. Pharmacists and health centers benefit from a low cost way to train patients how to use inhalers correctly, even if the patient could not afford to buy a sensor for continuous use with the inhaler. Biotechnology and pharmaceutical companies benefit by receiving a competitive advantage if they bundle the present invention with the companies' products and by receiving assurance that new drugs of the companies entered the patients (and were not lost of left in inhalation hardware due to device failures or patient errors) during pivotal clinical trials.

Most inhalers have a common interface between the device and the patient's mouth, e.g., an oval or round profile approximately 2-3 cm in diameter. The sensor of the present invention can in an exemplary embodiment be adapted to fit all types of inhalers, including, e.g., pressurized metered dose inhalers, dry powder inhalers, and aqueous based inhalers. The device can be usable even in cases when other auxiliary devices, such as, e.g., spacers, are used with a parent inhaler. Healthcare providers are expected to be amenable to the present invention's sensor solution to device misuse.

In an exemplary embodiment, the sensor can be added by the physician to a prescription that also has on the prescription an inhaled pharmaceutical product (similarly to prescribing “Spacer—use as directed” in conjunction with inhalers). Since the sensor does not contain any drug, and is not associated with a specific product, it is believed that use of the invention does not require any collaboration or consent of the inhaler manufacturer, and would be approvable by the US Food and Drug Administrations 510(k) process.

Optical sensing of the present invention as adapted does not present an obstruction to the egress of airborne particles, and does not alter the emitted drug dose or particle size. Optical sensing according to the present invention depends on the ability of the aerosol particles to block the transmission of light—the sensor is insensitive to the nature of the particle, and is usable with all drugs and pulmonary drug delivery systems. Optical sensing according to the present invention can be used in a range of bench-top laboratory instruments for other purposes including, for example, determining the size or concentration of airborne particles), and can be use in industrial applications including, for example, monitoring smoke emission. The technology is available, mature and can be integrated according to the present invention in readily miniaturized form.

Another exemplary embodiment of the present invention can be adapted to detect partial doses or inhalation of the incorrect drug. The present invention provides an ability to detect medication error and amounts to a significant breakthrough in pulmonary delivery.

Further features and advantages of the invention, as well as the structure and operation of various embodiments of the invention, are described in detail below with reference to the accompanying drawings.

### BRIEF DESCRIPTION OF THE DRAWINGS

The foregoing and other features and advantages of the invention will be apparent from the following, more particular description of a preferred embodiment of the invention, as illustrated in the accompanying drawings wherein like reference numbers generally indicate identical, functionally similar, and/or structurally similar elements. The left most digits in the corresponding reference number indicate the drawing in which an element first appears.

FIG. 1 depicts an exemplary embodiment of a conventional metered dose inhaler (MDI) as could be coupled a dosing sensor according to the present invention;

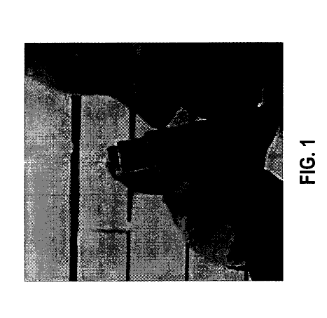

FIG. 2 depicts an exemplary embodiment of a unit dose sampling system as could be used in an exemplary embodiment of the present invention;

FIG. 3 depicts an exemplary embodiment of a schematic drawing illustrating modification of a sample collection tube to accommodate an optical sensing system in combination with an inhalation device according to the present invention;

FIG. 4 depicts an exemplary embodiment of a multistage liquid impinger and air flow controller according to the present invention;

FIG. 5 depicts an exemplary diagram illustrating an exemplary embodiment of a light sensing technology;

FIG. 6 depicts an exemplary embodiment of an infrared-based combustible gas detector that could also be used to detect emissions in an exemplary embodiment of the present invention; and

FIGS. 7A and 7B collectively depict exemplary diagrams illustrating an exemplary embodiment of a radiation sensing ionization chamber technology.

### DETAILED DESCRIPTION OF AN EXEMPLARY EMBODIMENT OF THE PRESENT INVENTION

A preferred embodiment of the invention is discussed in detail below. While specific exemplary embodiments are discussed, it should be understood that this is done for illustration purposes only. A person skilled in the relevant art will recognize that other components and configurations can be used without parting from the spirit and scope of the invention.

An exemplary embodiment of the present invention includes an inexpensive and compact optical/radiation sensing technology that is capable of detecting low concentrations of airborne particles.

Optical sensors are available based on several light sources operating at numerous wavelengths. Off-the-shelf sensors, which are inexpensive, and compact can be acquired and integrated according to the present invention. Conventional sensors can be can be adapted, or can be reengineered into a format suitable for use.

Detection Sensitivity

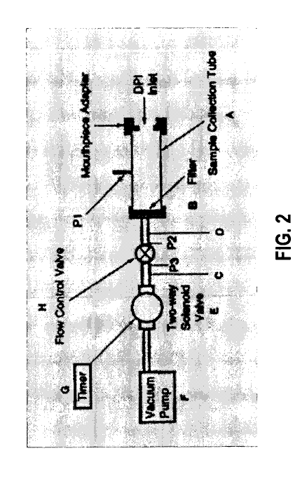

An exemplary embodiment of the present invention can include a sensor system that detects low concentrations of airborne particles just beyond the mouthpiece of most commercially available pulmonary delivery devices. Various types of MDI, DPI and nebulizers can be used. Detectability of solid particle or liquid droplet aerosols can be available. A test apparatus as illustrated in FIG. 2, can simulate flow rate, volume and duration of typical patient inhalations when used in combination with commercially available inhaled drug products. The test apparatus can ensure detection of an aerosol once diluted by patient inspiration, and when moving quickly in the inhaled air stream.

Experiments can be conducted using a modification of a commercially available aerosol testing system (such as, e.g., a unit dose sampling apparatus, available from Copley Scientific, of Nottingham, U.K. as depicted in FIG. 2). FIG. 2 depicts the unit dose sampling system of an exemplary embodiment of the present invention including a vacuum pump F coupled to a two-way solenoid valve E that can include a timer G. The solenoid valve can in turn be coupled to a flow control valve H via a coupler C. Flow control valve H can be coupled to a sample collection tube A via a coupler D, as shown. The vacuum tube pump is used in the test apparatus to simulate patient inhalations. The sample collection tube A can include a filter B, a mouthpiece adapter, and a DPI outlet into which an inhaler can be inserted.

Using the vacuum pump F, airflow rates of 15, 30, 60, 90, 120 and 180 L/min (representative of typical patient flow rates inhaled by patients using inhalers) can be set using the flow control valve H, and two-way solenoid valve E that is controlled by timer G for determining duration and volume of each inhalation. Sensitivity can be investigated at inhaled volumes of, e.g., between 500 ml and 4 liters, and inhalation durations of, e.g., between 0.5 to 4 seconds. Test inhalers can be placed in the DPI outlet and the manufacturers' instructions can be followed to release a dose of drug. In another exemplary embodiment, the test system can be replaced with a system which allows fast and slow rates of rise in flow rate to more precisely mimic patient inhalations.

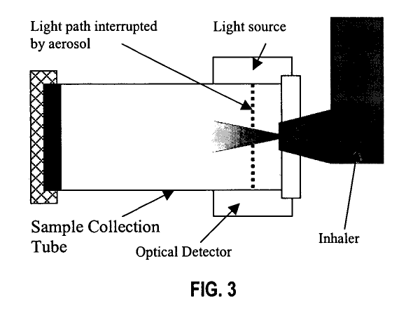

The dose sampling apparatus of FIG. 2 can be adapted by drilling two holes opposite one-another in the sample collection tube directly in front of the DPI outlet (as depicted for example in FIG. 3). FIG. 3 illustrates a schematic diagram showing modification of a sample collection tube to accommodate an optical sensing system apparatus according to the present invention. The optical sensing apparatus of FIG. 3 as shown is coupled to an inhaler. The device shown in FIG. 3 includes a light source (transmitter) and a detector (receiver) for receiving the light source. The optical sensor can include a sample collection tube coupled to the light source and detector. The sensor can be positioned close to where it would be located in a patient use situation, and can permit cleaning of optics between tests. Various optical systems can be included in the present invention including, e.g., ones based on a conventional bulb, diode and laser diode operating in the visible and infrared (IR) spectrum. Appropriate electronics can be provided for each type of transmitter and detector (some output voltage, others current as the response signal) so that detector output can be analyzed and/or recorded. An exemplary embodiment of a sensor can be used to acquire signals for further analysis. Sensors can include, e.g., an optical sensor, or a radiation sensor. An exemplary radiation sensor can include a sensor similar to the sensor used in a smoke detector. Of course other sensors could be used such as, e.g., radiation sensors or other optical sensors than those described in the present disclosure.

Because the test apparatus can be semi automated, data collection can be relatively efficient. For this reason a full factorial experimental design can be used with multiple (such as, e.g., 10) repeat measures of each treatment. This can allow precise mapping of sensitivity of each detector tested under each simulated inhalation condition. The ability to detect passage of emitted aerosol with a high degree (such as, e.g., 90%) of certainty can be used as a definition of a successful “detection event”. The sensor system that generates the largest number of successful detection events can be advanced to another stage of testing.

Detection of Incorrect Inhaler Use

The device of the present invention can also be adapted to detect inappropriate modes of inhaler use by patients. Testing can be conducted using the testing apparatus described previously with representative MDI, DPI and aqueous based aerosol generation systems to simulate exhalation into the sensor and no breathing at all. In other experiments, breathing can be simulated, but drug release can be deliberately prevented (e.g., by not actuating pressurized MDI or placing empty capsules or blisters in DPI systems). Tests can look for evidence that the sensor correctly identifies that the drug is not exiting the mouthpiece using the detection event criteria described above.

An exemplary embodiment of the present invention can also include a sensor mounting assembly adapted to have a maximum interior dimensions that would not alter performance of an original inhaler device.

The sensor of the present invention can be adapted in a form of a ring that can slide over, and can slightly extend the mouthpiece of a pharmaceutical inhaler that can be used in combination with the sensor. To be capable of approval by the food and drug administration (FDA) the “modified mouthpiece” can be demonstrated to not alter performance of the original pharmaceutical inhaler product. For this reason, the emitted dose (i.e., total mass of drug leaving the mouthpiece) and emitted fine particle dose (i.e., mass of drug contained in particles less than 5 micrometers—those most likely to avoid capture in the mouth and reach deep into the lung) can be measured in the presence and absence of several modified mouthpieces used in conjunction with each type of inhaler. Inhalers containing albuterol sulfate and beclomethasone propionate can, e.g., be tested since these drugs are delivered using multiple inhaler platforms.

In an exemplary embodiment of the present invention, several extensions, such as, e.g., 1, 2 and 3 cm extensions with, e.g., smooth internal transitions from an inhaler mouthpiece can be molded out of a substance such as, e.g., silicon. In one exemplary embodiment, the device can be as compact as possible, but in other exemplary embodiments, the device can of a larger size. The extensions can be placed between the inhaler mouthpiece and a DPI outlet of a collection tube A. The apparatus shown in FIG. 2 and described above can be used for collection of emitted drug doses. In the testing apparatus, drug doses can be collected on the filter B, that can be downstream of the inhaler used with or without extension tubes. Flow rates of, e.g., 30 and 90 liters/min with simulated inhalation volumes of, e.g., 2 and 4 liters can be tested for each drug. Following disassembly, the contents of the filter can be quantitatively extracted with a solvent and the drug content can be determined with an appropriate high pressure liquid chromatography (HPLC) assay. Sensitive HPLC assays can be established for multiple drugs. Experiments can be replicated several times and a variance analysis can be performed and least significant difference testing at the 5% significance level can be performed to look for differences in emitted dose of drug from the different sized mouthpiece extensions, and results can be compared with results obtained in the absence of any extension.

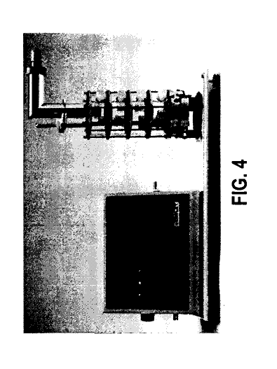

Using a multistage liquid impinger (MSLI) and air flow controller apparatus as shown in FIG. 4, a duplicate series of experiments can be performed in which the unit dose sampling system of FIG. 2 can be replaced by the MSLI. Albuterol in MDI and DPI platforms can be evaluated using the apparatus. Flow rates of, e.g., 30 and 90 liters/min with simulated inhalation volumes of, e.g., 2 and 4 liters can be tested. Drug can be recovered from each calibrated stage of the MSLI and can be quantitated using a validated HPLC assay in order to compare the fine particle drug dose and mass median aerodynamic diameter in the presence and absence of mouthpiece extensions. The same statistical procedure described above can be used to look for any difference in performance.

Potential obstacles to effective use of the present invention include that following multiple uses of an inhaler, aerosol may accumulate on optics, reducing detection sensitivity. It is believed that such aerosol accumulation can be minimized by positioning the optics out of the path of the emitted aerosol. If the aerosol accumulation persists deflectors can be incorporated in another exemplary embodiment on the interior of the sensor housing to avoid aerosol accumulation directly on the optics. The deflectors can be aerodynamically designed to minimize any interference by the deflectors. The testing methods overviewed above can be complemented by detection of presence of false positive detections, e.g., by tampering with the valve mechanism, and seeing if the sensor indicates that an incomplete dose was emitted.

The optical sensor system of the present invention can further include miniaturized electronics and optics.

Test results using a test apparatus as shown in Table 1 and 2 can be performed. For example, Ventolin, i.e., albuterol 90 ug per dose, available from GlaxoSmithKline, a widely prescribed pressurized metered dose inhaler product, can be connected to a typical spacer such as, e.g., AEROCHAMBER® brand of spacer available from Monaghan Medical of Plattsburgh, N.Y., USA, as described in the spacer manufacturers' instructions for patient use. A light beam from an inexpensive optical trigger such as, e.g., a Wavesensor optical trigger, intended for use with a 35 mm camera, can be directed through a small opening in a tube connected to the spacer mouthpiece. The trigger can be used at its midpoint sensitivity setting. Air can be “inhaled” through the tube to simulate a patient inhalation through the spacer using a vacuum pump drawing, e.g., 60 liters a minute of room air. Tests can be conducted in the absence of a simulated inhalation. A display on the optical sensor can indicate when the aerosol particles broke the light beam and “triggered” the device. The Ventolin MDI can be fired, e.g., three times at one minute intervals, and the number of times the sensor is triggered can be recorded. To test sensitivity of the detection mechanism, the spacer can be used with and without an exhalation prevention valve in place. Results of such a test are shown in Table 1 below.

TABLE 1
Detection of Emitted Aerosol from a Spacer/
MDI combination in the Presence
and Absence of a Simulated Inhalation

| Test Condition | Number of Detected | Actuations |
|---|---|---|
| No airflow / | 0 | out of 3 |
| Valve in mouthpiece | | |
| 60 L/min airflow / | 3 | out of 3 |
| No valve in mouthpiece | | |
| 60 L/min airflow / | 2 | out of 3 |
| Valve in mouthpiece | | |

While crude, the data indicates that even for a low dose drug such as albuterol, emitted into a large volume spacer (to further dilute the aerosol plume) it is feasible to detect aerosol as the aerosol leaves the mouthpiece of the spacer. This is true in the presence of and absence of an exhalation prevention valve, suggesting that the system works in several common modes of patient use. Detection of higher dose inhaled drugs, or those used without spacers should be even more reliable since the aerosol concentration in the mouthpiece is higher. Conversely, in the absence of a simulated inhalation, when the MDI is actuated, the sensor is not triggered. This is a common patient misuse scenario (actuation without inhaling) that is detected by the present invention.

In order to explore limits of detector sensitivity, an experiment can be conducted in which Ventolin MDI is fired in the direction of a camera sensor light beam from increasing distances. The results are shown in Table 2.

TABLE 2
Detection of Emitted Aerosol from a Spacer/
MDI combination in the Presence
and Absence of a Simulated Inhalation

| Test Condition | Number of Detected | Actuations |
|---|---|---|
| Ventolin fired at sensor from 5 cm | 3 | out of 3 |
| Ventolin fired at sensor from 10 cm | 3 | out of 3 |
| Ventolin fired at sensor from 15 cm | 3 | out of 3 |
| Ventolin fired at sensor from 20 cm | 3 | out of 3 |
| Ventolin fired at sensor from 25 cm | 3 | out of 3 |
| Ventolin fired at sensor from 30 cm | 2 | out of 3 |
| Ventolin fired at sensor from 35 cm | 0 | out of 3 |
| Ventolin fired at sensor from 40 cm | 0 | out of 3 |

The observed data gives a strong indication that simple optical sensors are able to detect low concentrations of aerosol moving at speeds that can be expected during patient inhalation. The observed results highlight the feasibility of an optical dose sensor.

Any of various conventionally available sensors and future sensors can be used with the present invention. In an exemplary embodiment, an optical sensor can be used. In another exemplary embodiment, a radiation sensor can be used.

In one exemplary embodiment of the apparatus of the present invention light can be transmitted by a light source. The light transmitted can be a visible spectrum light, ultraviolet, infrared spectrum, or other nonvisible spectrum light. An optical sensor, or light detector can sense the transmission. In an exemplary embodiment a light sensor can be used that operates similarly to a photoelectric sensor in a smoke detector.

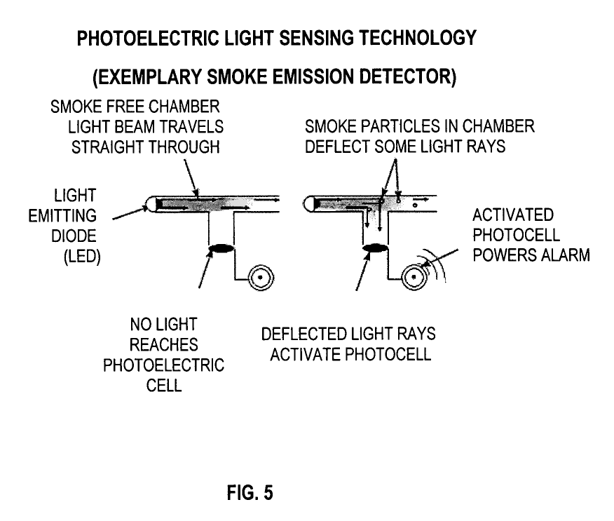

FIG. 5 depicts an exemplary diagram illustrating an exemplary light sensing technology. Specifically, FIG. 5 illustrates photoelectric technology works. Photoelectric technology smoke alarms, for example, use a T-shaped chamber fitted with a light-emitting diode (LED) and a photocell. The LED sends a beam of light across the horizontal bar of the chamber. The photocell sits at the bottom of the vertical portion of the chamber. The photo cell generates a current, when exposed to light.

The diagram of FIG. 5 illustrates photoelectric technology. Under normal, smoke-free conditions, the LED beam moves in a straight line, through the chamber without striking the photo cell. When smoke enters the chamber, smoke particles deflect some of the light rays, scattering them in all directions. Some of the smoke reaches the photocell. When enough light rays hit the photocell, they activate it. The activated photocell generates a current. The current powers the alarm, and the smoke alarm is activated. As will be apparent to those skilled in the art, such a photoelectric detector can be similarly used to detect light transmitted by a source, or other transmissions to detect emissions emitted from an emitter such as an inhaler, as described in the present invention.

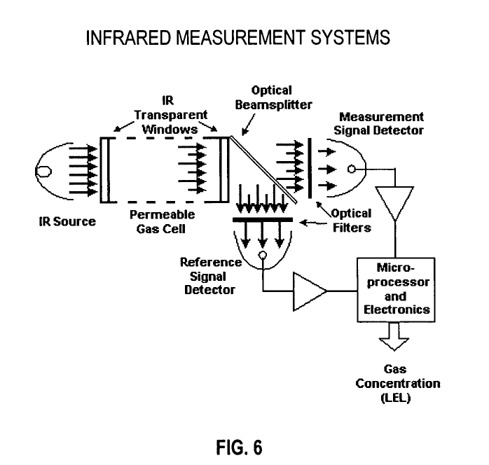

In another alternative exemplary embodiment, an optical sensor can be used. FIG. 6 illustrates an exemplary optical detector. Specifically, FIG. 6 depicts an infrared-based combustible gas detector that could also be used to detect other emissions as those detected in the present invention. Optical detectors for combustible gas detection provide an alternative to conventional pellistor-based combustible gas detectors. One reason for their popularity is ease of implementation in safety instrumented systems (SIS) intended for high availability and safety integrity levels (SIL).

The fundamental operation of optical gas detectors is described further below. The historical method of detecting combustible gases involved sensors based on pellistor beads. Pellistor based sensors are susceptible to significant undetected sources of failure. Primary of these are catalyst poisoning and flame arrestor plugging, either of which prevent the sensing of gas. In the above example, it is easy to see how a covert pellistor failure could remain undetected for much longer than ten (10) hours as the only way to detect a failure is to periodically check with gas. Absence of undetected failure modes is an intrinsic characteristic of infrared gas detectors that provides an advantage over catalytic beads. Failure modes of an optical gas detector are overt and instantly signaled so that repair can be initiated immediately.

Infrared gas detectors use variations on the basic measurement scheme outlined in the illustration of FIG. 6. As depicted, an infrared source illuminates a volume of gas that has diffused into a measurement chamber. The gas absorbs certain of the infrared wavelengths as the light passes through it while others pass through unattenuated. The amount of absorption is related to the concentration of the gas. The amount of absorption can be measured by a set of optical detectors and subsequent electronics. The change in intensity of the absorbed light can be measured relative to the intensity of light at a non-absorbed wavelength. A microprocessor can compute and report gas concentration from the absorption. When there is no gas present the signals for the reference and active channel sensors are balanced. When there is combustible gas present, there is a predictable drop in the output from active channel sensor because the gas is absorbing light. A fault condition is encountered in the case of dirty optics or a weak and failing light source. The former scenario is a trigger to perform routine maintenance and the latter is an indicator that preventive maintenance should be scheduled. In either case, an instrument can continue to faithfully measure gas concentration up until the situation degrades to an untenably low signal level. These maintenance situations can be flagged with digital field communication and asset management and predictive maintenance programs. When failed components are in the measurement loop, i.e., a sensor fails or light source fails, signals on one or both sensor channels will fall to zero.

Alternatively, a radiation sensor can be used to sense transmission of the emissions such as, e.g., an alpha emission. Alpha particles can be emitted and detected. For example americium or palladium can be used and detected.

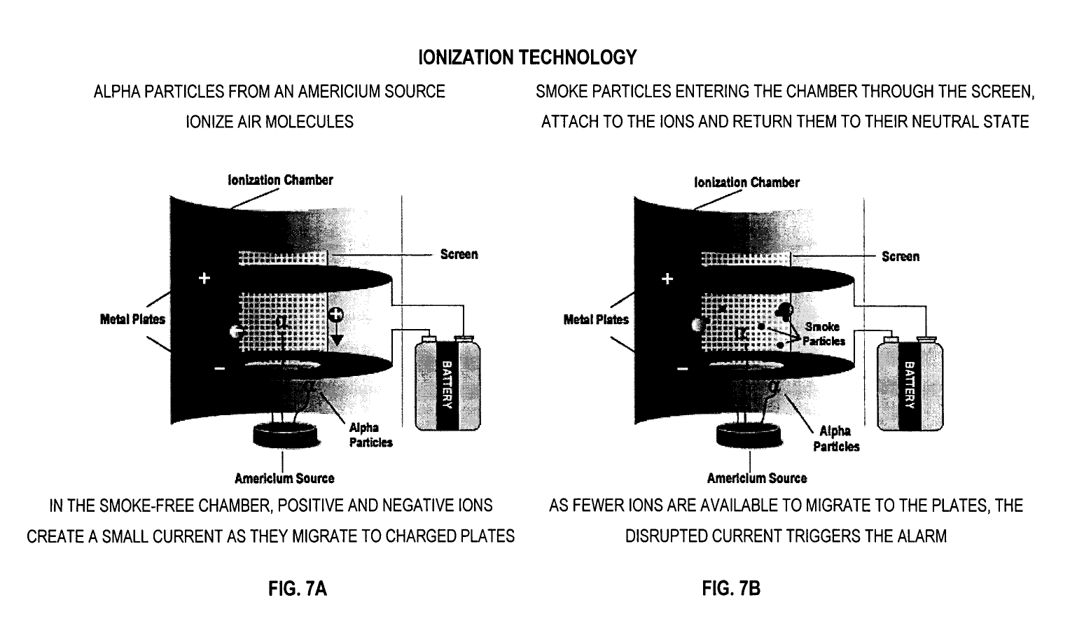

FIGS. 7A and 7B collectively depict exemplary diagrams illustrating an exemplary radiation sensing ionization chamber technology. Specifically, FIGS. 7A and 7B illustrate ionization technologies such as those used in ionization sensor smoke alarms. Ionization sensor smoke alarms contain a small amount of radioactive material, such as, e.g., americium, embedded in a gold foil matrix within an ionization chamber. The matrix can be made by rolling gold and americium oxide ingots together to form a foil approximately one micrometer thick. This thin gold-americium foil can then be sandwiched between a thicker (˜0.25 millimeter) silver backing and a 2 micron thick palladium laminate. This is thick enough to completely retain the radioactive material, but thin enough to allow alpha particles to pass.

The ionization chamber can include two metal plates a small distance apart. One of the plates can carry a positive charge, the other a negative charge. Between the two plates, air molecules—made up mostly of oxygen and nitrogen atoms—can be ionized when electrons are kicked out of the molecules by alpha particles from the radioactive material (alpha particles are big and heavy compared to electrons). The result is oxygen and nitrogen atoms that are positively charged because they are short one electron; the free electrons are negatively charged.

The diagrams depicted in FIGS. 7A and 7B illustrate how ionization technology works. The positive atoms flow toward the negative plate, as the negative electrons flow toward the positive plate. The movement of the electrons registers as a small but steady flow of current. When smoke or other emissions enter the ionization chamber, the current is disrupted as the emission particles attach to the charged ions and restore them to a neutral electrical state. This reduces the flow of electricity between the two plates in the ionization chamber. When the electric current drops below a certain threshold, the alarm is triggered.

FIG. 7A illustrates alpha particles from an americium source ionizing air molecules. In an emission-free chamber, positive and negative ions create a small current as they migrate to charged plates. FIG. 7B illustrates emission particles entering the chamber through the screen, attaching to the ions and returning the ions to a neutral state. As fewer ions are available to migrate to the plates, the disrupted current triggers activation of, e.g., a smoke alarm, or detection of particles.

While various embodiments of the present invention have been described above, it should be understood that they have been presented by way of example only, and not limitation. Thus, the breadth and scope of the present invention should not be limited by any of the above-described exemplary embodiments, but should instead be defined only in accordance with the following claims and their equivalents.

1. An apparatus for providing positive confirmation of inhaled drug delivery by attenuation of light or radiation at point-of-use comprises:
a sensing mechanism that senses emissions from an inhaler, wherein said sensing mechanism is adapted to be coupled to the inhaler.

2. The apparatus of claim 1, wherein said emissions comprise at least one of:
aerosol;
drug particles;
propellants;
flavors;
colors; and
other emissions.

3. The apparatus of claim 1, further comprising:
a tube adapted to be coupled at an outlet of the inhaler.

4. The apparatus of claim 1, wherein said sensing mechanism comprises at least one of:
an optical sensing device; and
a radiation sensing device.

5. The apparatus of claim 4, further comprising a tube adapted for coupling to an outlet of the inhaler, wherein said optical sensing mechanism comprises at least one of:
a light source coupled to said tube; and
a detector coupled to said tube.

6. The apparatus of claim 5, wherein said light source transmits light along a path intersecting a path of an emission emitted from the inhaler, and wherein said detector receives the light from said light source.

7. The apparatus of claim 6, wherein said light comprises at least one of:
a visible spectrum light;
ultraviolet spectrum light; and
an infrared spectrum light.

8. The apparatus of claim 4, wherein said radiation sensing device operates in similar fashion to a smoke detector in sensing said emissions.

9. The apparatus of claim 3, wherein said outlet is a dry powder inhaler (DPI) outlet.

10. The apparatus of claim 1, wherein said apparatus further comprises:
an inhaler.

11. The apparatus of claim 10, wherein said inhaler comprises at least one of:
a dry power inhaler (DPI);
a pressurized metered dose inhaler (MDI); and
a pressurized MDI-spacer combination.

12. The apparatus of claim 3, wherein said tube comprises a cylinder having a geometrically shaped cross-section comprising at least one of:
a circular cross-section;
a rectangular cross-section;
a square cross-section;
a polygon cross-section;
a symmetrical cross-section;
an asymmetric cross-section; and
a cross-section suitable for coupling with specific types of inhalers.

13. The apparatus of claim 1, wherein the apparatus further comprises:
a deflector that deflects emissions to avoid emission accumulation.

14. The apparatus of claim 13, wherein said deflector deflects emissions from at least one of said sensing mechanism, a light source, and a detector.

15. The apparatus of claim 4, further comprising a tube adapted for coupling to an outlet of the inhaler, wherein said radiation sensing mechanism comprises at least one of:
a radiation source coupled to said tube; and
a detector coupled to said tube.

16. The apparatus of claim 15, wherein said radiation source transmits radiation along a path intersecting a path of an emission emitted from the inhaler, and wherein said detector receives the radiation from said radiation source.

17. The apparatus of claim 6, wherein said light comprises:
alpha radiation.

18. An apparatus for providing positive confirmation of emission delivery by attenuation at point-of-use comprises:
sensing means for sensing emissions from an emitter, and
coupling means for coupling said sensing means to the emitter.

19. The apparatus of claim 18, wherein the emitter comprises an inhaler.

20. A method for providing positive confirmation of emission delivery at point-of-use comprising:
sensing emissions from an emitter.
